(19) United States
(12) Patent Application Publication
(10) Pub. No.: US 2009/0012543 A1
(43) Pub. Date: Jan. 8, 2009

(54) DEVICE AND METHOD FOR ANASTOMOSIS

(75) Inventor: Hassan Kansoul, Norsborg (SE)

Correspondence Address: DRINKER BIDDLE & REATH (DC), 1500 K STREET, N.W., SUITE 1100, WASHINGTON, DC, 20005-1209, US

(73) Assignee: Prozeo Vascular Implant AB, Stockholm (SE)

(21) Appl. No.: 12/160,660

(22) PCT Filed: Jan. 12, 2006
(86) PCT No.: PCT/SE06/00051

§ 371 Date: Jul. 11, 2008

(51) Int. Cl. A61B 17/11

(52) U.S. Cl. 606/153

(57) ABSTRACT

The invention relates to an anastomosis device for anastomising two vessel parts together. First and second attachment units are included. They are connectable to each other and attachable to a respective vessel part. According to the invention each attachment unit has a vessel receiving surface with an area that has a roughness with a large number of small sharp projections arranged to partly penetrate the wall of the received vessel part without reaching through said wall. The invention also relates to a method for anastomosis and to a use of the invented anastomosis device.

### FIELD OF INVENTION

The present invention in a first aspect relates to an anastomosis device for anastomising two vessels parts or other tubular organs together, the device including first and second attachment units attachable to a respective vessel part and connectable to each other, each attachment unit having a through-hole defining a longitudinal axis of the attachment unit, and each attachment unit having a vessel receiving surface.

In a second aspect the invention relates to a method for anastomising two vessel parts together by attaching first and second attachment units to a respective vessel part and connecting the attachment units to each other, each attachment unit having a through-hole defining a longitudinal axis of the attachment unit.

In a third aspect the invention relates to a use of the invented device.

In this application definitions like axial, radial, tangential are related to said longitudinal axis.

### BACKGROUND OF THE INVENTION

Anastomosis is the surgical joining biological tissue to create an internal communication between them. Vascular surgery often involves creating an anastomosis between blood vessels to create or restore blood flow to essential tissues. At present, the majority of all vascular anastomosis procedures are performed by conventional hand suturing. Suturing an anastomosis is time consuming and difficult. It is important that each anastomosis provide a smooth and open flow path and the attachment must be leak free under relatively high pressures. A completely leak free anastomosis is not always achieved on the first try. Consequently, there is a frequent need to re-suture the anastomosis to close any leaks or remove any flow interruptions that are detected.

Attempts has been made to provide devices and methods that allow more reliable anastomosis and which require shorter operation time.

Examples of such devices and methods are disclosed in U.S. Pat. No. 3,258,012, U.S. Pat. No. 4,917,090 and U.S. Pat. No. 6,524,322.

U.S. Pat. No. 3,258,012 discloses a method of connecting blood vessels using a pair of needle discs having alternately placed projecting needles and receiving bores on an annular base member, and forceps having clamping jaws forming, when clamped together, an annular groove in which the discs may be positioned, comprising directing a first blood vessel through a bore of forceps and a needle disc positioned in the groove thereof and securing the outer walls of the blood vessel to the needles projecting therefrom, directing a second blood vessel through a second forceps through the bore thereof and the bore of a needle disc held thereby and anchoring the walls to the needles projecting therefrom, positioning the two of said forceps so that the needles of one disc align with the openings defined in the other, and pressing the forceps together to cause the needles of one disc to enter the bores of the other and to deflect the needles around the exterior walls of opposing discs to interlock said discs and said blood vessels.

U.S. Pat. No. 4,917,090 discloses fasteners for the joining of blood vessels or tubular organs. The fasteners are shaped in such a manner that they are retained in a novel clamping device until the joining is accomplished. The fasteners comprise at least two couplable rings having axially directed pins and intermediate holes distributed about their centers, and a shoulder portion formed along the periphery of at least one of the rings for fitting at least one of the rings into the clamping device.

U.S. Pat. No. 6,524,322 discloses an anastomotic device for joining vessels, in the first place joining one need of a graft vessel to a target vessel at an opening made in the wall thereof. The anastomotic device comprises a tubular body on which an outer flange, which comes into contact with the outside of the wall of the target vessel around the opening, and an inner flange, which comes into contact with the inside of the wall of the wall of the target vessel around the opening, are arranged. The inner flange is made up of a number of arms which are able to move from an extended position, located in the extension of the tubular body, under the influence of a pretension into a position extending in the lateral direction with respect to the tubular body, after the pretension has been released, in order to form the inner flange. In FIGS. 18-22 in particular end-to-end joining is disclosed.

In the prior art disclosures the attachment of each unit to a respective vessel part is performed by folding the vessel part over an end surface of the attachment part which end surface has projections like needles or the like that penetrate the wall of the vessel part.

Penetration through the wall results in that the needle reach out into the interior of the connected vessel. The holes thereby created by the needles might be sources for infection or other complications for the patient.

The object of the present invention is to provide an anastomosis device in which this drawback is overcome and which thus avoids this damage to the vessel part that might cause this harm to the patient.

### DESCRIPTION OF THE INVENTION

This object has according to the first aspect of the invention been achieved in that an anastomosis device according to the preamble of claim 1 includes the specific features that the vessel receiving surface of at least one of the attachment units has a roughness with a large number of small projections arranged to partly penetrate the wall of the received vessel without reaching through said wall.

Since the projections do not completely reach through the wall they will not reach the inside of the connected vessel and there will be no openings facing the interior of the vessel that could cause infection or the like. With a large number of such small projections the connection will be sufficiently strong although each projection reaches only partly through the wall. By a large number in this application is meant between 50 and 10 000.

According to a preferred embodiment the height of the projections is in the range of 0.05 to 1.00 mm, preferably in the range of 0.1 to 0.5 mm.

Within these ranges the height of the projections is sufficient to attain a sufficient attachment force and simultaneously avoid that the projections reach the other side of the wall.

According to a further preferred embodiment the vessel receiving surface is located on an axially directed end wall facing the other attachment unit when the attachment units are connected to each other.

Attaching the vessel wall to an axially directed surface gives a more secure attachment than any other alternative. When the attachment units are connected to each other the vessel walls will be squeezed between the attachment units such that a very reliable connection will be achieved.

According to a further preferred embodiment said surface is divided into a plurality of separate areas.

This allows an optimal distribution of the active surface for attachment such that the area that is provided with projections can be minimized thereby minimizing the manufacturing costs for the device.

According to a further preferred embodiment the end wall in a section perpendicular to the longitudinal axis has a non-planar shape.

A non-planar shape offers a possibility to optimize the end wall surface for receiving a good connection.

According to a further preferred embodiment the non-planar shape is the same for both units.

Thereby it can be attained that the end walls abut each other only in particular areas, so that the contact force can be controlled and optimized.

According to a further preferred embodiment the shape of the end wall of the first attachment unit is complementary to the shape of the second attachment unit such that the end walls fit into each other.

This strengthens the connection since relative movement between the end walls is prevented by the locking effect of the parts that fit into each other.

According to a further preferred embodiment each unit includes a cylindrical wall defining said through-hole, and in a section through the longitudinal axis the cylindrical wall is connected to the end wall by a curved arc portion, preferably a circular arc portion.

Thereby the end wall and the cylindrical inner wall meet each other without any sharp edges that otherwise would entail the risk of damaging the vessel wall when folded out on the end wall. The curved arc minimize the risk of any cutting effect on the vessel wall.

According to a further preferred embodiment at least one of the attachment units includes means for varying its circumferential lengths.

Thereby an easy adaption to variations in the size of the vessel part in question is attained.

According to a further preferred embodiment at least one of the attachment units includes two relative to each other rotatable parts, having the longitudinal axis as the axis of rotation, said connection means being arranged on a first of said parts and said vessel receiving surface being arranged on a second of said parts.

By allowing a relative rotation between a part of the attachment unit that is connected to the other attachment unit and a part to which the vessel wall is attached it can be avoided that torsion tension occurs in the vessels.

According to a further preferred embodiment each of the attachment unit has a non-circular shape in a section perpendicular to the longitudinal axis.

This embodiment is particularly useful when end to side or side to side anastomosis is concerned. If a circular attachment unit is applied to a side opening of a vessel there will be a pattern of tensions in the vessel wall that tend to deform the vessel such that it in part will be narrower. This is avoided by using an attachment unit that is non-circular, e.g. having an elliptic cross section.

According to a further preferred embodiment at least one of the attachment units is deformable.

Thereby the shape of the attachment units can be modified and adapted to the shape of the vessel parts to which they are intended to be attached.

The above described preferred embodiments of the invented device are specified in the claims dependent from claim 1.

According to the second aspect of the invention the object has been achieved in that a method of the kind specified in the preamble of claim 19 includes the specific measures of applying the wall of the respective vessel part to a vessel receiving surface of the respective attachment unit such that a large number of small sharp projections on the receiving surface partly penetrate the wall of the vessel without reaching through said wall.

By the invented method and the preferred embodiments thereof advantages are gained corresponding to the advantages of the invented device and the preferred embodiments thereof which have been described above.

### BRIEF DESCRIPTION OF THE DRAWINGS

FIG. 1 is a perspective view of a first unit of a device according to the invention.

FIG. 2 is a perspective view of a second unit of a device according to the invention.

FIG. 3 is a simplified end view of the attachment unit of FIG. 1.

FIG. 4 is a perspective view of a part of FIG. 3.

FIG. 5 is a view corresponding to FIG. 3 illustrating an alternative example.

FIG. 6 is a perspective view of a part of the attachment unit of FIG. 1 according to a modified example.

FIG. 7 is a simplified end view of the attachment unit of FIG. 1 according to another modified example.

FIG. 8-10 are simplified sections through the attachment units when being connected and illustrate various shape of their axial end walls.

### DETAILED DESCRIPTION OF ADVANTAGEOUS EXAMPLES OF THE INVENTION

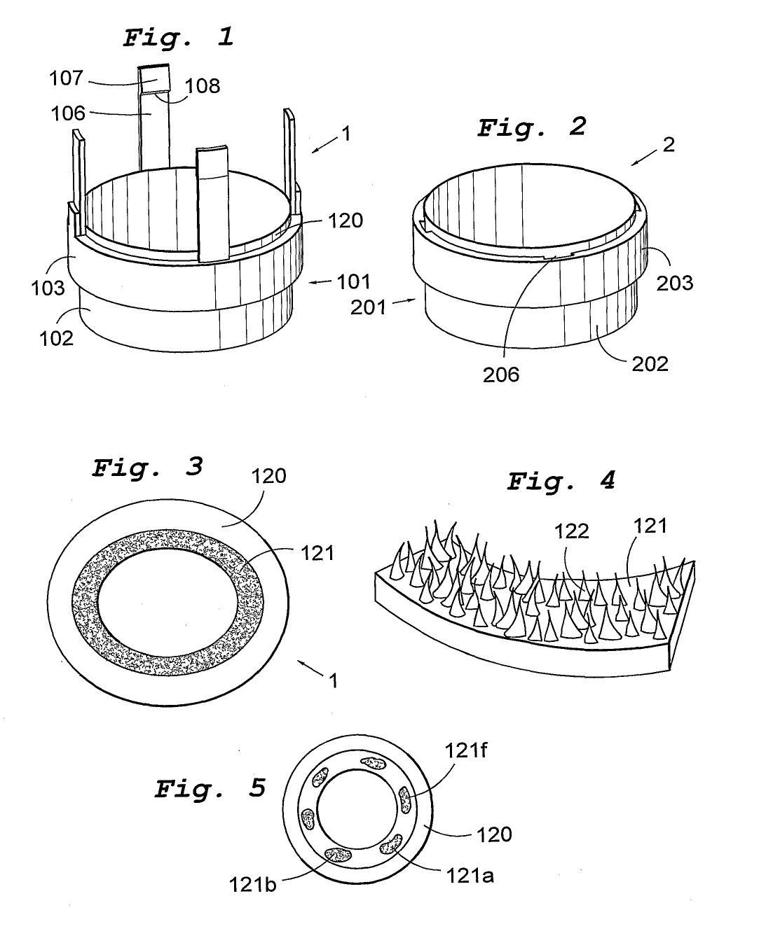

The anastomosis device consists of first 1 and second 2 attachment units, where FIG. 1 depicts the first unit 1 and FIG. 2 the second unit 2. The attachment units are preferably made of titanium. They can also be made of a resorbable material, partly or completely.

FIG. 1 thus is a perspective view of the first attachment unit 1. The unit has a body 101 that is generally cylindric. The body has a rear part 102 and a front part 103, the front part 103 having a slightly larger outer diameter than the rear part 102. In the illustrated example the unit is circular cylindric, but other cylindrical shapes, e.g. elliptical cylindric are possible.

Means for attaching the units to a vessel end and means for connecting the unit to the second unit extend substantially axially from the front edge of the front part 103.

The means for attaching the unit to a blood vessel consists of a large number of short sharp projections located on a vessel receiving surface 120. The arrangement of these are explained more in detail in relation to FIGS. 3 and 4.

The means for connecting the unit 1 to the second unit 2 consists of four axially extending members 106, in the illustrated example shaped as tongues. Also the number of tongues can vary but in most cases 4 tongues is appropriate. Each tongue 106 is flexible in the radial direction for allowing the connection with the second unit 2 as will be described later. Each tongue 106 at its outer end is provided with a radially offset part 107 creating a shoulder 108 where it joins the inner part of the tongue 106.

FIG. 2 is a perspective view of the second attachment unit 2. Also this unit has a body 201 that is generally cylindric and has a rear part 202 and a front part 203, where the front part 203 has slightly larger outer diameter than the rear part 202. The diameter of the body 201 corresponds to the diameter of the body of the first part 1.

At the edge of the front part 203 a number of axially directed openings 206 are provided. The number of the openings 206 corresponds to the number of tongues 106 of the first unit 1, and the cross sectional shape of each opening 206 generally corresponds to the cross sectional shape of each tongue 106.

The units 1 and 2 can be connected to each other by means of the tongues 106 and openings 206. The connection is established by moving the units 1, 2 axially towards each other while the ends of the tongues 106 are inserted in the openings 206. Due to the flexibility of the tongues 106 in the radial direction they can snap into a locking position in the openings 206 so that the units are axially locked to each other.

FIG. 3 schematically illustrates the vessel receiving surface 120 located on the axial end wall of the first attachment unit 1.

On the radially inner part of this surface 120 there is an area 121 that is rough, the roughness being created by a large number of short and sharp projections.

In FIG. 4 these projections 122 are enlarged illustrated. The height of the projections 122 is in the range of 0.05-1.0 mm, preferably in the range of 0.1-0.5 mm.

When attaching the unit 1 to a vessel, the vessel end is drawn through the through-hole in the middle in a direction up from the plan of the paper. The vessel end is then folded outwardly across the surface 120. The part of the vessel wall which thereby contacts the area 121 will be partly penetrated by the projections 122 such that the vessel is attached to the unit 1.

The height of the projections 122 in the prescribed range results in that the projections do not reach all the way through the vessel wall.

FIG. 5 illustrates an alternative example of arranging the vessel receiving surface 121. In this embodiment the area with the projections is constituted by a number of limited areas 121a to 121f.

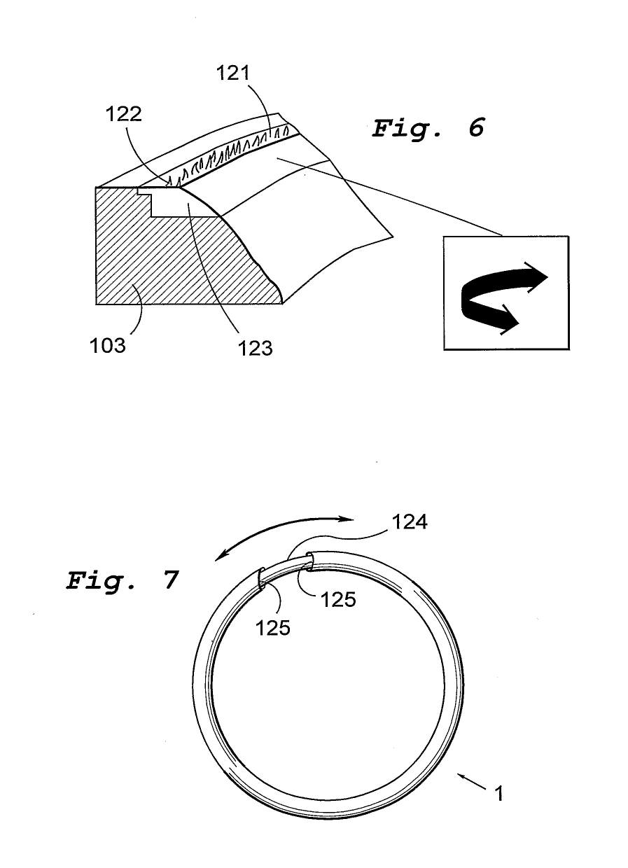

FIG. 6 illustrates an example of the invention where the vessel receiving area 121 with the projections 122 is located on a separate ring member 123 which is rotatable in relation to the rest of the front part 103 of the first attachment unit 1. The connection means for connection to the second attachment unit is fixed to the rest of the front part 103.

FIG. 7 in a simplified end view illustrates a further alternative example of the attachment unit 1. The attachment unit is partly hollow and splited such that two openings 125 are formed. An adjusting member 124 extend telescopically into these openings. Thereby the diameter of the attachment unit 1 can be varied.

FIGS. 8 to 10 are simplified sections through the attachment units 1, 2 when being connected to each other. The two vessel ends extend vertically from the top and bottom, respectively in each of the figures and are folded out in the area when the vessel receiving surfaces 120, 220 on the circumferential end walls of each attachment unit 1 and 2 face each other.

From e.g. FIG. 8 it can be seen that the central through-hole of the attachment unit 1 meets the vessel receiving surface 120 through a section 126 that extends along a circular arc in cross section.

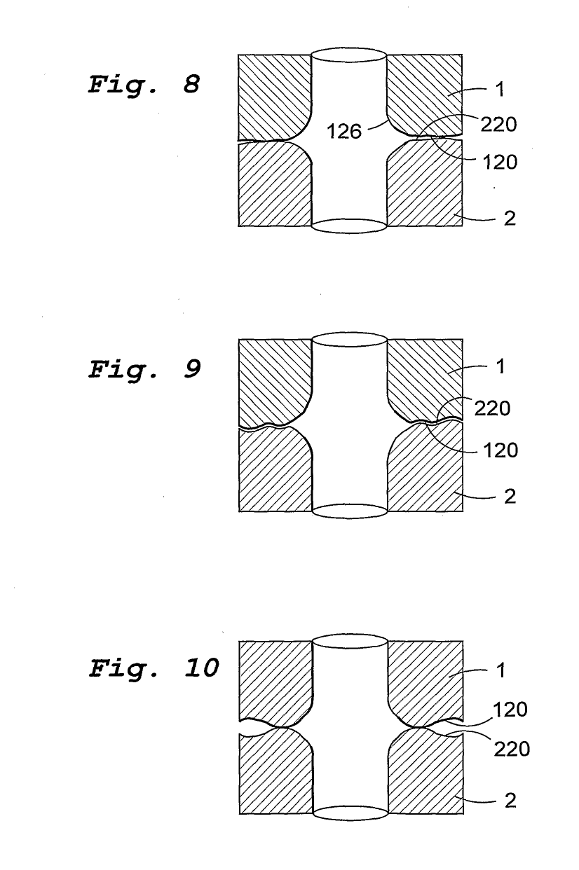

In FIG. 9 the vessel receiving surfaces 120, 220 of the two attachment units have an ondulated shape such that they fit into each other.

In FIG. 10 the vessel receiving surfaces 120, 220 of the two attachment units have the same non-planar shape, resulting in a distinct area when they abut each other.

It is to be understood that the different examples described above with reference to the attachment unit 1 also apply to the attachment unit 2.

1. An anastomosis device for anastomising two vessel parts or other tubular organs together, the device including first and second attachment units attachable to a respective vessel part and connectable to each other by connection means on each unit, each attachment unit having a through-hole defining a longitudinal axis of the attachment unit and each attachment unit having a vessel receiving surface wherein the vessel receiving surface of at least one of the attachment units includes an area having a roughness with a large number of small sharp projections arranged to partly penetrate the wall of the received vessel part without reaching through said wall, which area is located on an axially directed end wall facing the other attachment unit when the attachment units are connected to each other.

2. An anastomosis device according to claim 1, wherein the height of the projections is in the range of 0.05 to 1.0 mm.

3. An anastomosis device according to claim 1, wherein said area extends circumferentially on said axially directed end wall.

4. An anastomosis device according to claim 1, wherein said area is divided into a plurality of separate areas.

5. An anastomosis device according to claim 1, wherein said end wall in a section perpendicular to the longitudinal axis has a non-planar shape.

6. An anastomosis device according to claim 5, wherein said non-planar shape is the same for both units.

7. An anastomosis device according to claim 5, wherein the shape of the end wall of the first attachment unit is complementary to the shape of the end wall of the second attachment unit (2) such that the end walls fit into each other.

8. An anastomosis device according to claim 1, wherein each unit includes a cylindrical wall defining said through-hole and in that in a section through the longitudinal axis the cylindrical wall is connected to the end wall by a curved arc portion.

9. An anastomosis device according to claim 1, wherein at least one of the attachment units includes means for varying its circumferential length.

10. An anastomosis device according to claim 1, wherein at least one of the attachment units includes two relative to each other rotatable parts having the longitudinal axis as the axis of rotation, said connection means being arranged on a first of said parts and said area being arranged on a second of said parts.

11. An anastomosis device according to claim 1, wherein each of the attachment units has a non-circular shape in a section perpendicular to the longitudinal axis.

12. An anastomosis device according to claim 1, wherein at least one of the attachment units is plastically deformable.

13. A method for anastomising two vessel parts or other tubular organs together by attaching first and second attachment units to a respective vessel part and connecting the attachment units to each other, each attachment unit having a through-hole defining a longitudinal axis of the attachment unit, the method comprising applying the wall of the respective vessel part to a vessel receiving surface of the respective attachment unit such that a large number of small sharp projections on the vessel receiving surface partly penetrate the wall of the vessel without reaching through said wall, which protections are located on an axial wall facing the other attachment unit when the attachment units are connected to each other.

14. A method performed by using a device according to claim 1.

15. The use of a device according to claim 1 for anastomising two vessels to each other.

16. The use according to claim 15, where said vessels are blood vessels.

17. An anastomosis device according to claim 2, wherein the height of the projections is in the range of 0.1 to 0.5 mm.

18. An anastomosis device according to claim 8, wherein the curved arc portion is a circular arc portion.
